(19) United States
(12) Patent Application Publication
(10) Pub. No.: US 2013/0268036 A1
(43) Pub. Date: Oct. 10, 2013

(54) Apparatus and Method of Treating a Vein with Heat Energy

(71) Applicants: Sean Morris, Chesterfield, MO (US); Joseph M. Tartaglia, Morgan Hill, CA (US); Daniel Recinella, Queensbury, NY (US); Kimberly McCarthy, Ballston Spa, NY (US); Lowell S. Kabnick, Far Hills, NJ (US)

(72) Inventors: Sean Morris, Chesterfield, MO (US); Joseph M. Tartaglia, Morgan Hill, CA (US); Daniel Recinella, Queensbury, NY (US); Kimberly McCarthy, Ballston Spa, NY (US); Lowell S. Kabnick, Far Hills, NJ (US)

(21) Appl. No.: 13/802,293

(22) Filed: Mar. 13, 2013

Related U.S. Application Data
(60) Provisional application No. 61/620,334, filed on Apr. 4, 2012.

(51) Int. Cl. A61F 7/12

(52) U.S. Cl. CPC A61F 7/12
USPC 607/105

(57) ABSTRACT

A method of delivering therapy to a vein. In one embodiment, the method includes the following steps: inserting a structural sheath into the vein, the structural sheath being configured to prevent collapse of the vein due to spasm or via the administration of tumescent anesthesia; advancing a vapor delivery shaft into the catheter sheath; positioning a vapor delivery tip of the vapor delivery shaft distally of the catheter sheath; and delivering vapor to the vein through the vapor delivery tip to, e.g., shrink the vein with the vapor. The invention also includes a vapor delivery catheter system adapted to perform the method.

### CROSS REFERENCE TO RELATED APPLICATIONS

This application claims the benefit of U.S. Application No. 61/620,334, filed Apr. 4, 2012, the disclosure of which is incorporated by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to treatment of blood vessel disorders. More specifically, this disclosure relates to using vapor therapy to reduce an inner diameter of a vessel in the leg of a patient.

### BACKGROUND

The human venous system of the lower limb consists essentially of the superficial venous system and the deep venous system with perforating veins connecting the two systems. The superficial system includes the great saphenous, small saphenous and the lateral saphenous systems. The deep venous system includes the anterior and posterior tibial veins which unite to form the popliteal vein, which in turn becomes the femoral vein when joined by the short saphenous vein.

The venous systems contain numerous one-way valves for facilitating blood flow back to the heart. Venous valves are usually bicuspid valves, with each cusp forming a sack or reservoir for blood when, under pressure, forces the free surfaces of the cusps together to prevent retrograde flow of the blood and allows antegrade flow to the heart. When an incompetent valve is in the flow path of retrograde flow toward the foot, the valve is unable to close because the cusps do not form a proper seal and retrograde flow of blood cannot be stopped.

Incompetence in the venous system can result from vein dilation, which causes the veins to swell with additional blood. Separation of the cusps of the venous valve at the commissure may occur as a result. The leaflets are stretched by the dilation of the vein and concomitant increase in the vein diameter which the leaflets traverse. Stretching of the leaflets of the venous valve results in redundancy which allows the leaflets to fold on themselves and leave the valve open. This is called prolapse, which can allow reflux of blood in the vein. Eventually the venous valve fails, thereby increasing the strain and pressure on the lower venous sections and overlying tissues. Two venous diseases which often involve vein dilation are varicose veins and chronic venous insufficiency.

The varicose vein condition includes dilatation and tortuosity of the superficial veins of the lower limb, resulting in unsightly protrusions or discoloration, ‘heaviness’ in the lower limbs, itching, pain, and ulceration. Varicose veins often involve incompetence of one or more venous valves, which allow reflux of blood from the deep venous system to the superficial venous system or reflux within the superficial system.

Current varicose vein treatments include invasive open surgical procedures such as vein stripping and occasionally vein grafting, venous valvuloplasty and the implantation of various prosthetic devices. The removal of varicose veins from the body can be a tedious, time-consuming procedure and can be a painful and slow healing process. Complications including scarring and the loss of the vein for future potential cardiac and other by-pass procedures may also result. Along with the complications and risks of invasive open surgery, varicose veins may persist or recur, particularly when the valvular problem is not corrected. Due to the long, arduous, and tedious nature of the surgical procedure, treating multiple venous sections can exceed the physical stamina of the physician, and thus render complete treatment of the varicose vein conditions impractical.

Newer, less invasive therapies to treat varicose veins include intralumenal treatments to shrink and/or create an injury to the vein wall thereby facilitating the collapse of the inner lumen. These therapies include sclerotherapy, as well as catheter, energy-based treatments such as laser, Radio Frequency (RF), or resistive heat (heater coil) that effectively elevate the temperature of the vein wall to cause collagen contraction, an inflammatory response and endothelial damage. Sclerotherapy, or delivery of a sclerosant directly to the vein wall, is typically not used with the larger trunk veins due to treatment complications of large migrating sclerosant boluses. Laser energy delivery can result in extremely high tissue temperatures which can lead to pain, bruising and thrombophlebitis. RF therapy is typically associated with lengthy treatment times, and resistive heater coil treatments can be ineffective due to inconsistent vein wall contact (especially in larger vessels). The catheter based treatments such as laser, resistive heater coil and RF energy delivery also typically require external vein compression to improve energy coupling to the vein wall. This is time consuming and can again lead to inconsistent results. In addition, due to the size and/or stiffness of the catheter shaft and laser fibers, none of these therapies are currently being used to treat tortuous surface varicosities or larger spider veins. They are currently limited in their use to large trunk veins such as the great saphenous vein (GSV). Tortuous surface varicosities are currently treated with sclerotherapy and ambulatory phlembectomy, while larger spider veins are currently only treated with sclerotherapy.

### SUMMARY OF THE DISCLOSURE

One aspect of the invention provides a method of delivering therapy to a vein. The method includes the following steps: inserting a structural sheath into the vein, the structural sheath being configured to prevent collapse of the vein due to spasm or via the administration of tumescent anesthesia; advancing a vapor delivery shaft into the catheter sheath; positioning a vapor delivery tip of the vapor delivery shaft distally of the catheter sheath; and delivering vapor to the vein through the vapor delivery tip to, e.g., shrink the vein with the vapor.

In some embodiments, the vapor may be generated remotely from the vapor delivery shaft, and in other embodiments, the vapor may be generated within the vapor delivery shaft.

In some embodiments, prior to the delivering step, the structural sheath is retracted proximally along the vapor delivery shaft to expose a portion of the vapor delivery shaft. The exposed portion of the vapor delivery shaft may form a hot zone having a length of approximately 5 cm to 15 cm. In some embodiments, the retracting step includes the step of retracting the structural sheath until a portion of the sheath engages a fitting extending from the vapor delivery shaft.

Some embodiments of the invention include the step of pulling the structural sheath and the vapor delivery shaft proximally along the vein during the delivering vapor step. Such embodiments may also include the step of maintaining a relative position between the catheter sheath and the vapor delivery shaft during the pulling step.

Another aspect of the invention provides a method of treating a vessel. The method includes the following steps: inserting a structural sheath into a vessel to be treated; applying tumescent anesthesia around the vessel; preventing the vessel from collapsing due to the tumescent anesthesia with the structural sheath; advancing a vapor delivery catheter into the sheath to position delivery vapor ports of the catheter within the vessel; and delivering vapor through the vapor delivery ports to treat the vessel by, e.g., shrinking the vessel.

Some embodiments of this aspect of the invention include the step of pulling the structural sheath and the vapor delivery catheter proximally within the vein during the delivering step. The method may also include the steps of stopping the pulling of the structural sheath and the vapor delivery catheter when the catheter reaches a feeder vein to be treated; steering the delivery vapor ports into or towards the feeder vein; and delivering vapor through the vapor delivery ports into the feeder vein to treat the feeder vein.

Yet another aspect of the invention provides a vapor delivery catheter system having a structural sheath adapted to be inserted into a vein; and a vapor delivery shaft adapted to be surrounded by the structural sheath, the vapor delivery shaft including a vapor delivery port at a distal end of the shaft and a fitting at a proximal end of the shaft, the fitting being adapted to engage with the structural sheath when the vapor delivery port extends from a distal end of the sheath. In some embodiments, the fitting is further adapted to attach the sheath to the vapor delivery shaft so that the sheath and the shaft can be moved as a unit.

In some embodiments, the sheath has markings at the distal end of the sheath adapted to alert the user when the sheath is about to exit the entry site. The markings may be regularly spaced markings adapted to indicate depth of insertion of the sheath and at least one distal marking distinct from the regularly spaced markings at the distal end of the sheath.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is one embodiment of a vapor delivery catheter.

FIGS. 2A-2B show another embodiment of a vapor delivery catheter with a sheath slightly retracted to reveal a vapor delivery tip.

FIGS. 3A-3B show another embodiment of a vapor delivery catheter with a sheath retracted to reveal a vapor delivery tip and a hot zone of a catheter shaft.

FIG. 4 shows one embodiment of a vapor delivery catheter with a sheath having windows or openings.

FIGS. 5A-5E illustrate a method of treatment of a vessel.

FIG. 6 shows another embodiment of a vapor delivery catheter according to this invention.

FIG. 7 shows the vapor delivery catheter of FIG. 6 within a sheath with depth markings.

FIG. 8 is a close-up view of a portion of the vapor delivery catheter and sheath of FIG. 7.

FIG. 9 is another close-up view of a portion of the vapor delivery catheter and sheath of FIG. 7

### DETAILED DESCRIPTION

The disclosure relates generally to systems and their methods of use to treat venous insufficiency. More particularly, the invention relates to vapor treatment of a vein to reduce its inner diameter to minimize and/or eliminate blood flow through the vein. The therapy is generally used to divert the flow of blood from an insufficient vein to a vein that is sufficient.

The vapor treatments described herein can be used to treat any vein, such as trunk vessels (e.g., a great or small saphenous vein), sub-truncal veins (e.g., accessory vessels) or spider veins. The veins treated need not be varicose, however this is typically the case. The invention is not, however, limited to the treatment of the veins and the anatomical locations of the veins that are described herein. For example, the invention can be used to treat veins outside the leg region, such as abdominal varicosities, hemorrhoids, varicoceles, etc.

The treatments described herein generally include generating and delivering relatively high temperature (e.g., without limitation, greater than 37° C.) vapor through a delivery device to the lumen of a vein to reduce the inner diameter of the vein. A significant benefit of vapor delivery to reduce the lumen of the vessel is that it flows to the internal surfaces of the vein due to the increased pressure of the vapor and does not require external compression of the vein to enhance energy transfer of the device to the vein wall. Another significant benefit of the vapor delivery is the large amount of energy released in the transition of the vapor into the fluid phase. A further significant benefit of the vapor is that it is self-limiting in that it ceases to conduct heat to the vessel wall once temperature equilibrium has been reached between the vapor and the vessel wall. This is unlike other treatments which will continue to deliver energy to the tissue to the point of extensive thermal injury.

The vapor (such as steam) can be generated in a variety of locations in the system. For example, the vapor can be generated in a remote boiler or control console separate from the delivery device, within a handle or handpiece, or within the portion of the elongate member (such as a catheter) that is inserted into the vein. The vapor can be generated in any portion of the elongate member that is either inside or outside of the patient, for example.

This disclosure overcomes a major problem with the previously described catheter-based vapor system and others used in the clinic. Unlike other catheter based treatments such as laser, resistive heater coil and RF energy delivery, the catheter-based vapor system does not require external vein compression to improve energy coupling to the vein wall. The present disclosure improves the therapy by preventing the inadvertent compression and/or undesired spasm of the vein walls which hinders proper vapor dispensation. Embodiments described herein, including apparatus and methods of treating a vein with heat energy while preventing spasm, collapse or reduction in vein diameter prior to vapor therapy, provides this significant improvement.

As described above, any compression of the vessel or movement of the vein wall into the lumen prior and during treatment is not desired. This compression or movement can be due to: administration of tumescent anesthesia and the fluid volume delivery and associated needle stick; administration of anesthetic or cooling fluid (typically 0.9% normal saline) around or on top of the vein; ultrasound probe pressure; or vein spasm (due to irritation of the vein due to catheter placement; cold procedure room; needle stick for local anesthetic; or cold saline drip from catheter tip).

According to some embodiments, such vessel wall movement, luminal diameter reduction or distortion (e.g. flattening), or full lumen collapse (e.g., via administration of tumescent anesthesia) will not allow the vapor to freely flow from the vapor catheter tip exit ports out to the full internal luminal surface of the vein. In some instances, full lumen collapse over the catheter's vapor exit ports can completely block and prevent the delivery of vapor to a collapsed vessel. Therefore, preventing vessel collapse and administering the vapor to the full internal luminal surface of the vein to administer symmetrical and consistent heat energy is important to achieve proper vein shrinkage.

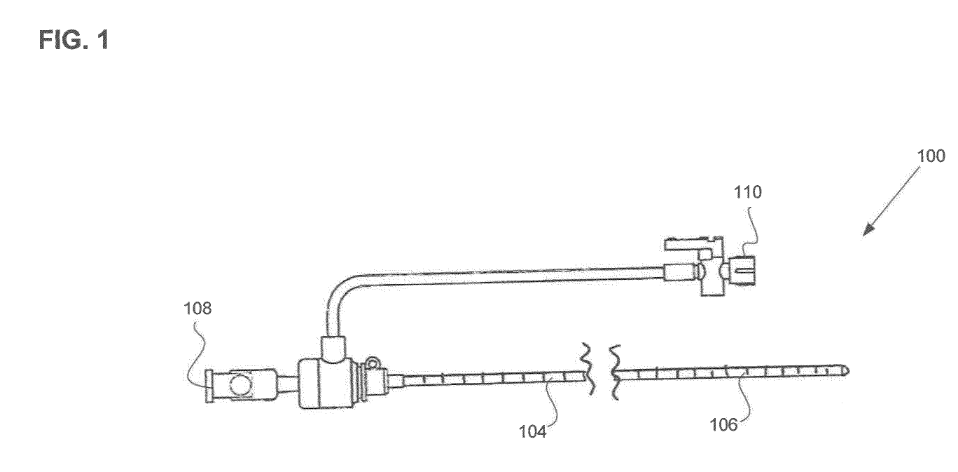

FIG. 1 illustrates a vapor catheter 100 configured to prevent luminal space reduction from occurring during vapor therapy treatment of blood vessels. In FIG. 1, the catheter 100 can include an elongate catheter shaft (not shown in FIG. 1) and a structural sheath 104 disposed over the shaft. The catheter can be, for example, a passive catheter, or in other embodiments, an active steerable catheter. The sheath can be retractable from the catheter shaft to reveal a vapor delivery tip (not shown in FIG. 1). The sheath can include markings 106 to facilitate the determination and control of pull back length and timing. In some embodiments, the markings can be spaced apart by a known distance (e.g., spaced apart by 1 cm). The markings can further include numbering or lettering. The sheath 104 is configured to have a structural strength sufficient to hold its shape and prevent collapse of the vessel (e.g., support the vessel) during or after application of tumescent anesthesia.

Catheter 100 can further include valve 108 and flush port 110. Valve 108 can be configured to couple the catheter to a control system and/or a vapor source. In some embodiments, the catheter receives vapor from an external source (e.g., a remote boiler), and in other embodiments the catheter generates vapor within the catheter itself. Flush port 110 can facilitate flushing the catheter with, for example, saline or another fluid/gas prior to or after therapy.

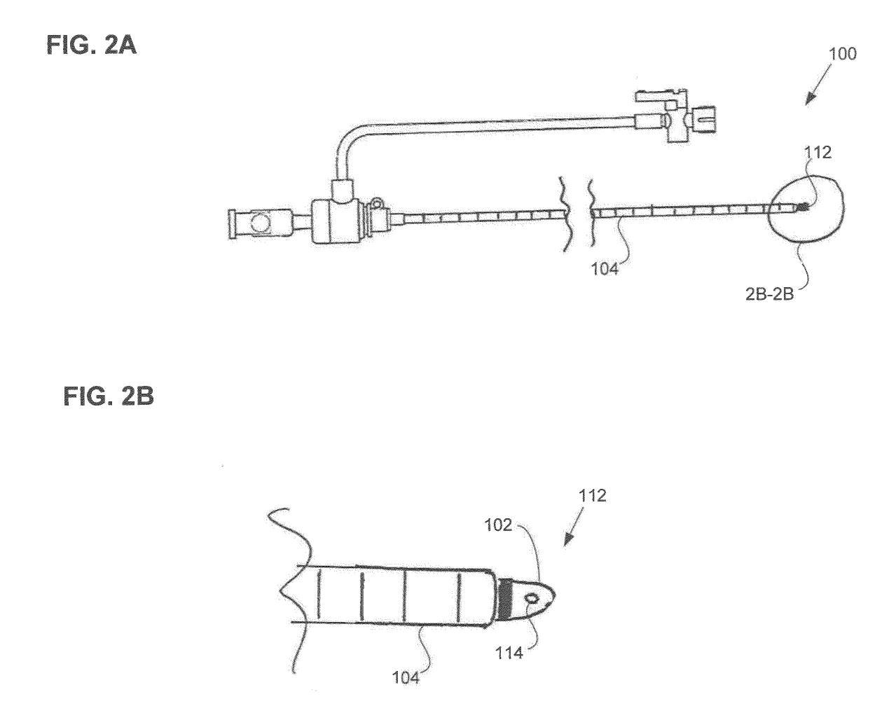

FIGS. 2A-2B illustrate one embodiment of vapor catheter 100 with structural sheath 104 slightly retracted proximally from the catheter shaft 102 to reveal a portion of vapor delivery tip 112 at the distal end of catheter shaft 102. In some embodiments, the sheath can be pulled back and locked in place to expose only the tip of the vapor catheter. FIG. 2A shows the catheter 100 with the sheath slightly retracted, and FIG. 2B is a close-up view of tip 112 revealing vapor port(s) 114. In the embodiment of FIGS. 2A-2B, the sheath 104 can be a non-heat resistant sheath or a heat-resistant sheath.

In the embodiment illustrated in FIGS. 2A-2B, the distal tip of the structural sheath can be positioned near the vapor ports of the vapor catheter to allow the sheath to prevent vessel walls from collapsing around the vapor ports when tumescent anesthesia is applied to the patient. By preventing the collapse of the vessel walls around the vapor catheter, vapor is allowed to escape from the vapor ports to treat the vessel. The sheath 104 can be non-heat resistant or made from conductive material, thereby allowing heat from the vapor catheter to propagate through the sheath and thermally damage the vessel walls.

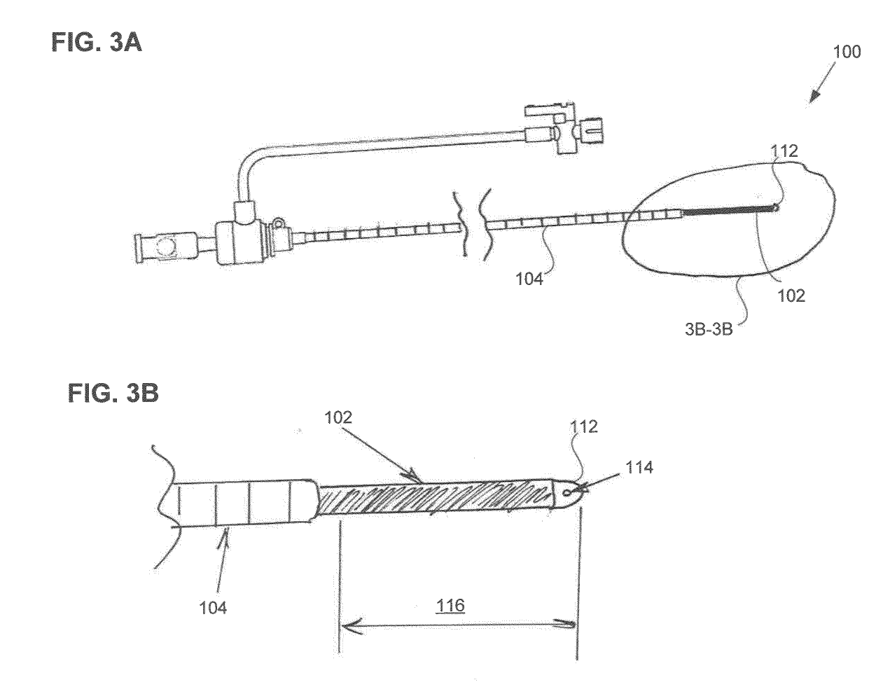

FIGS. 3A-3B illustrate another embodiment of the vapor catheter 100 with sheath 104 sheath 104 retracted further along shaft 102 to fully reveal vapor delivery tip 112 and a larger section of the shaft, illustrated as “hot zone” 116. The hot zone 116 can be the length of shaft 102 that is extended beyond the protective sheath and is configured to transmit heat energy to the vein to be treated. In the embodiment of FIGS. 3A-3B, the sheath 104 can be a heat resistant sheath and the shaft 102 can be a heat-emitting shaft, thus the sheath should be retracted back further along the shaft than in the embodiment of FIGS. 2A-2B.

As in the embodiment of FIGS. 2A-2B, the structural sheath 104 of FIGS. 3A-3B is configured to prevent collapse of the vessel walls around the vapor catheter due to tumescent anesthesia prior to delivery of vapor. The sheath 104 structurally supports the vessel walls, even after administration of tumescent anesthesia, thereby allowing vapor to escape from the catheter to treat the vessel. In FIGS. 3A-3B, the “hot zone” 116 of the shaft is configured to apply thermal energy to the vessel walls in addition to the vapor that exits the vapor ports of the catheter shaft.

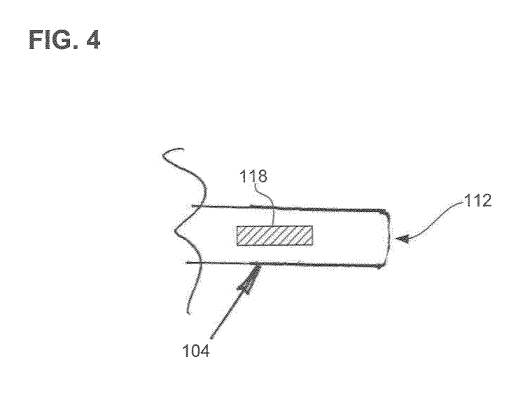

According to the embodiment of FIG. 4, a structural sheath 104 can include windows, holes, openings, or ports 118 to further allow propagation of vapor from the vapor delivery catheter to the vessel walls. In this embodiment, the window 118 is shown as a rectangular window in the structural sheath 104. However, in other embodiments, other types, sizes, and shapes of windows or ports can be used as long as the windows or ports are configured to allow vapor to propagate through the structural sheath and into the vessel walls. The vapor delivery catheter can be the same length as the structural sheath 104, so as to position vapor delivery tip 112 of the vapor catheter at or near a distal opening of the sheath. When vapor is delivered from the vapor catheter, the vapor can propagate through the window of the sheath an also through a distal opening in the sheath.

Methods of using the vapor delivery catheters described above will now be discussed. The specific clinical steps to be used are included for illustration purposes and are not specific constraints of this disclosure. First, a micro introducer kit can be used to gain venous access (not shown). The micro introducer kit can comprise, for example, a needle, 0.018 guide wire, and an introducer or, if desired, otherwise access using 18 g needle. Next, the guide wire can be advanced to the Sapheno Femoral Junction (SFJ).

Referring to FIG. 1, structural sheath 104 can be inserted through an access point into the vein to be treated up to the point at which the treatment is to be initiated. Next, tumescent anesthesia can be administered to the patient, as known, or cooling fluid such as saline can be applied to the vein or around the vein if desired. The structural sheath is configured to have sufficient structural strength to “stent” open the vein, to prevent vasospasm luminal reduction or other luminal reduction during this administration of tumescent anesthesia or cooling fluid around or on top of the vein. It is critical that the sheath prevent the vein from collapsing due to the administration of anesthesia so as to allow for vapor delivery in the following steps.

Next, catheter shaft 102 can be inserted into the sheath 104 and advanced distally until vapor ports 114 are positioned distal to the end of the sheath, as shown in FIG. 2A. The sheath can be withdrawn sufficiently to expose the vapor ports 114 or equivalent vapor dispenser orifices. Next, vapor energy can delivered from the catheter shaft through the vapor ports to the vein to be treated. The vapor energy can be generated in a remote boiler or can be generated within the catheter itself, for example. During vapor delivery, the sheath and catheter shaft can be simultaneously pulled back toward the vein access point in a continuous or step wise manner until the entire desired length of vessel is treated. The sheath and catheter shaft can be extracted from the vein at a predetermined rate and relative to the patient's specific anatomy (vein diameter, collateral flow, degree of closeness to the surface of the skin, etc.). The structural sheath 104 is configured to prevent compression or movement of the vessel before and during vapor therapy to allow the vapor to contact the full internal luminal surface of the vein, thereby providing symmetrical and consistent application of vapor energy to the vein to be treated.

As discussed above, FIGS. 3A-3B illustrate an embodiment when sheath 104 comprises a heat resistant sheath. In this embodiment, prior to the vapor delivery, the sheath is withdrawn from the catheter shaft sufficiently to expose the entire hot zone 116 of the catheter shaft 102. In one embodiment, the hot zone of the catheter shaft is approximately 10 cm, caused by withdrawal of the sheath by a minimum of 10 cm along the shaft. In other embodiments, the hot zone can be more or less than 10 cm. Once the sheath is properly positioned, vapor can be delivered to the vein as described above, by delivering vapor to the vein through the catheter shaft and vapor ports. The sheath and catheter shaft can be pulled backwards toward the vein access point as a unit during the vapor delivery (optionally using markings on the sheath) at a specified rate. The sheath 104 and the hot zone of the catheter are configured to prevent compression or movement of the vessel before and during vapor therapy to allow the vapor to contact the full internal luminal surface of the vein, thereby providing symmetrical and consistent application of vapor energy to the vein to be treated.

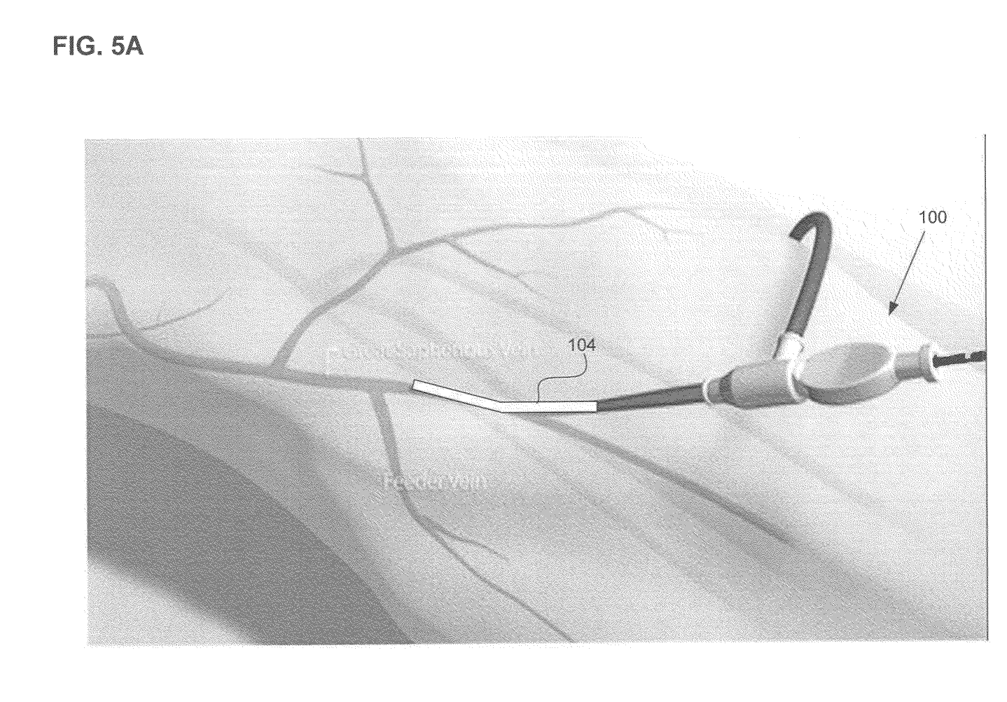

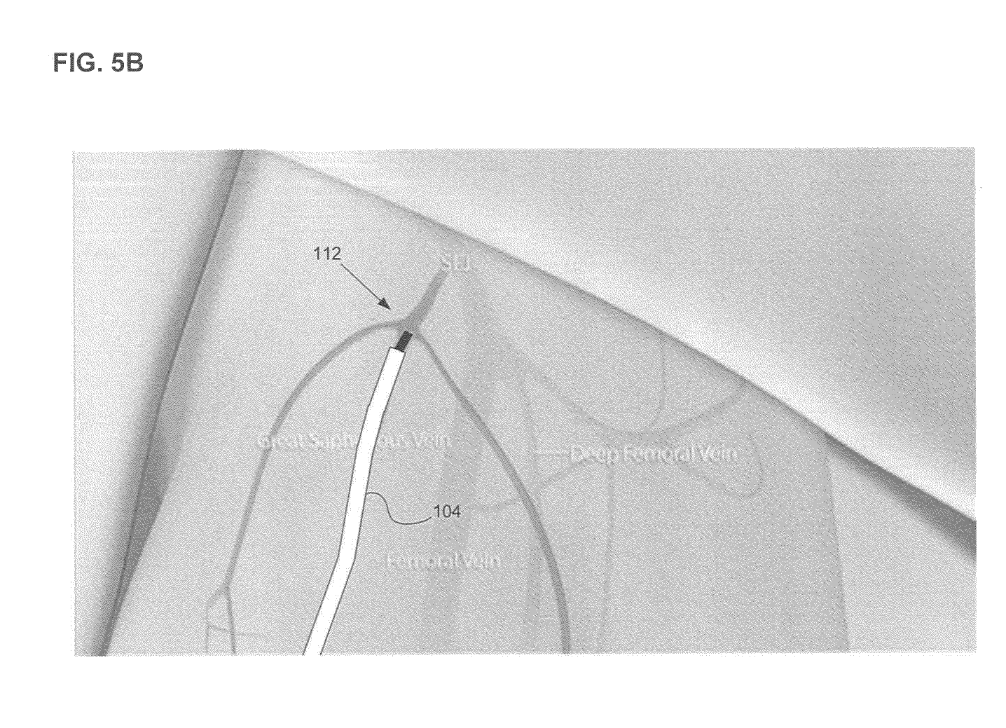

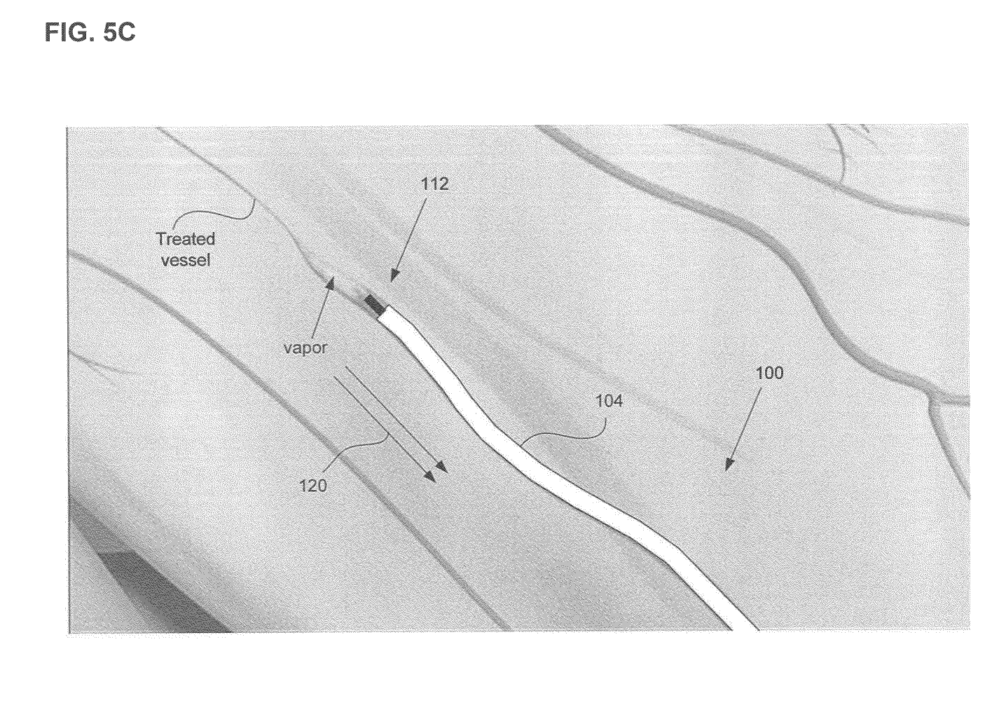

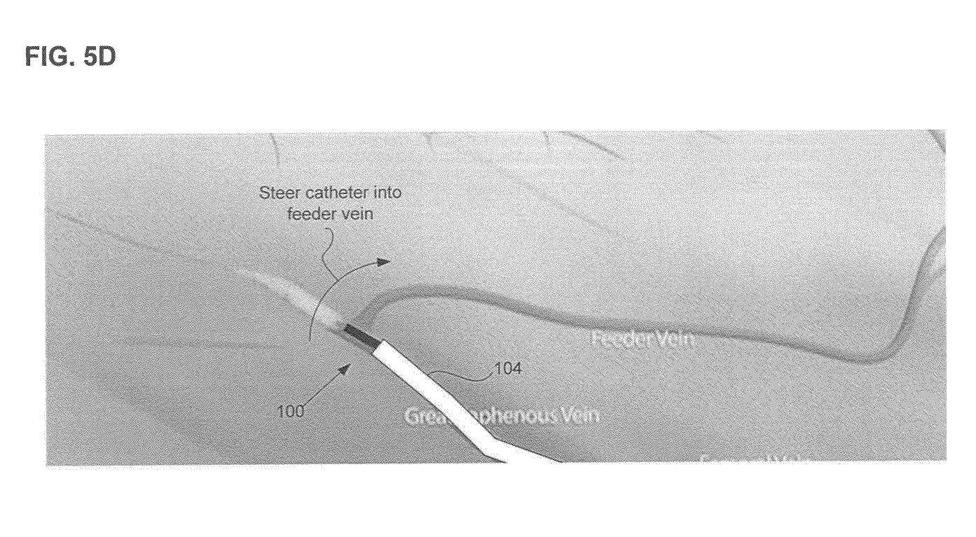

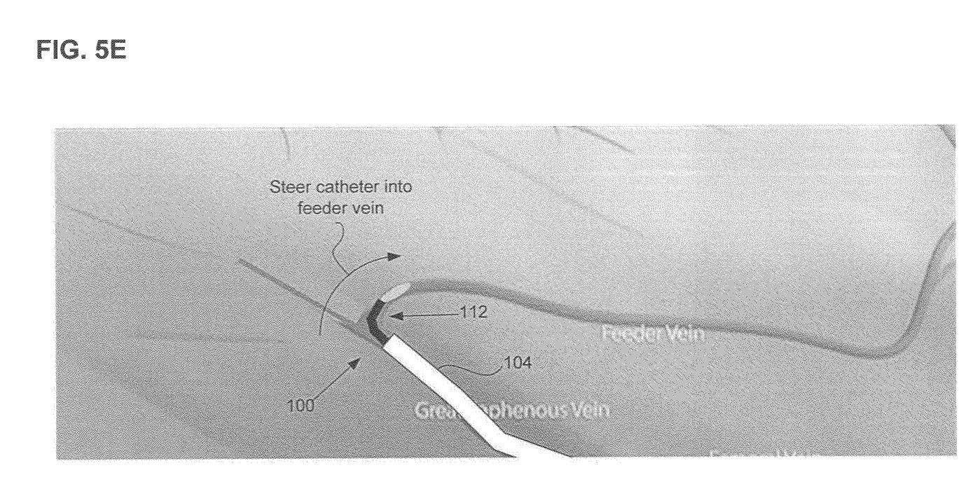

FIGS. 5A-5D illustrate a method of delivering therapy to a vein according to one embodiment. Referring to FIG. 5A, structural sheath 104 can be inserted into an access point of a vessel to be treated. In one embodiment, the access point for the sheath can be the greater saphenous vein (GSV) in the leg of a patient. In another embodiment, the access point can be the lesser saphenous vein.

Next, referring to FIG. 5B, the structural sheath 104 can be advanced along the vessel to be treated to the point where therapy is to be started. In one embodiment, the delivery tip is positioned at or near the sapheno femoral junction (SFJ). In other embodiments, the start point for placement of the delivery tip varies based on the condition of the vein to be treated. Proper positioning of the vapor catheter can be confirmed with ultrasound, for example. At this point in the method, the shaft of the vapor catheter can be inserted into the sheath and advanced towards the distal tip of the sheath. The vapor delivery ports 114 of the catheter can be advanced distally beyond the structural sheath, according to the embodiments of either FIGS. 2A-2B or FIGS. 3A-3B, as described above.

Next, tumescent anesthesia can be applied in or around the vessel to be treated, as known. The structural sheath is configured to prevent collapse of the vessel due to the tumescent anesthesia, or due to spasm of the vein. In some embodiments, tumescent anesthesia is not applied before therapy.

Referring now to FIG. 5C, vapor can be delivered from vapor delivery tip 112 to the vessel to be treated. The vapor can propagate out through the vapor catheter and the structural sheath. In some embodiments, the catheter shaft 102 and sheath 104 can be pulled proximally along the vessel towards the access point as vapor is being delivered from the catheter to the vessel. The catheter can be pulled along the length of the vessel to be treated, or pulled along at least a portion of the vessel that requires luminal diameter reduction. The rate of pulling can be determined based on the size of the vessel and the amount of vapor energy needed to treat the vessel. As shown, vapor delivery reduces the diameter of the treated vessel. The structural sheath 104, however, prevents collapse of the vein prior to vapor delivery.

FIGS. 5D-5E illustrate another embodiment of the method, useful for the treatment of feeder veins stemming from larger vessels in the leg. In some embodiments, it can be desirable to treat feeder veins with vapor therapy alongside treatment of the main vessel. As described above, the vapor catheter 100 can be pulled proximally along the vessel to be treated (such as the GSV) toward the catheter's access point into the vessel to deliver vapor to the vessel. When the catheter reaches the junction of the main vessel and a feeder vein, the catheter can be steered or turned towards and/or into the feeder vein to deliver vapor into the feeder vein, as shown in FIG. 5E. This can be accomplished, for example, by incorporating steering elements into the catheter, as described above, and actuating the steering elements to turn the vapor delivery tip of the catheter towards or into the feeder vessel. Vapor can be delivered into the feeder vein to treat the vein by reducing the diameter of the vein. The amount of time to deliver vapor to the feeder vein can vary based on the size/diameter of the feeder vein.

The vapor delivered by the vapor delivery catheter of this invention may be generated remote from the catheter and delivered to the catheter shaft or it may be generated within the catheter itself, such as in the catheter handle or catheter shaft. Details of one suitable manner of generating vapor within the catheter may be found in US Patent Publ. No. 2011/0264176, the disclosure of which is incorporated herein by reference.

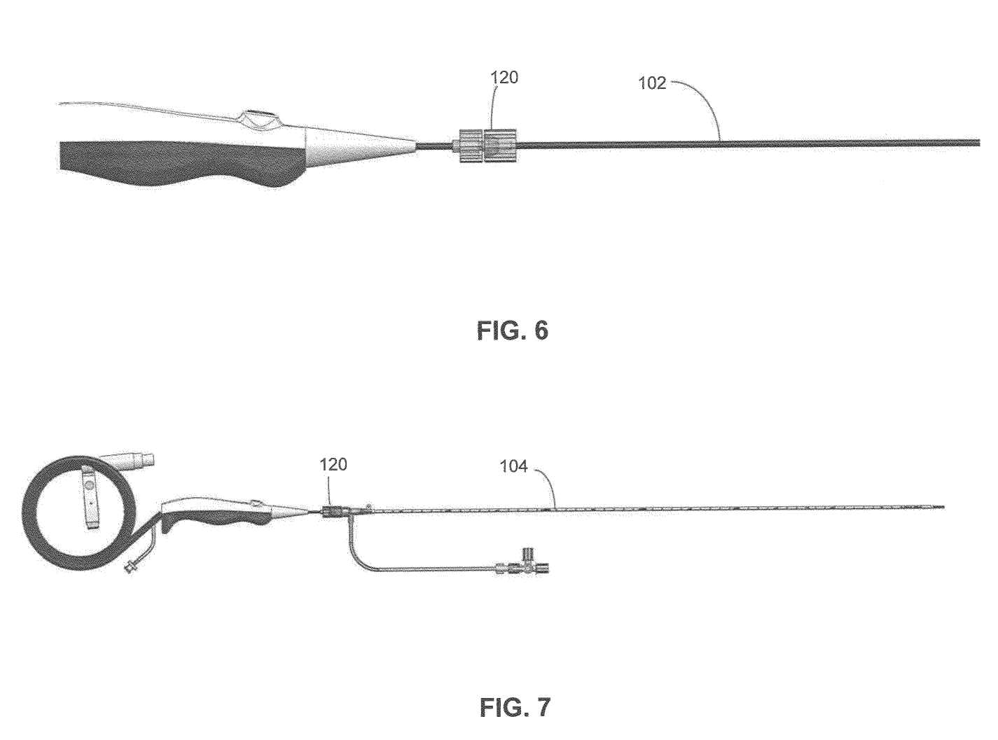

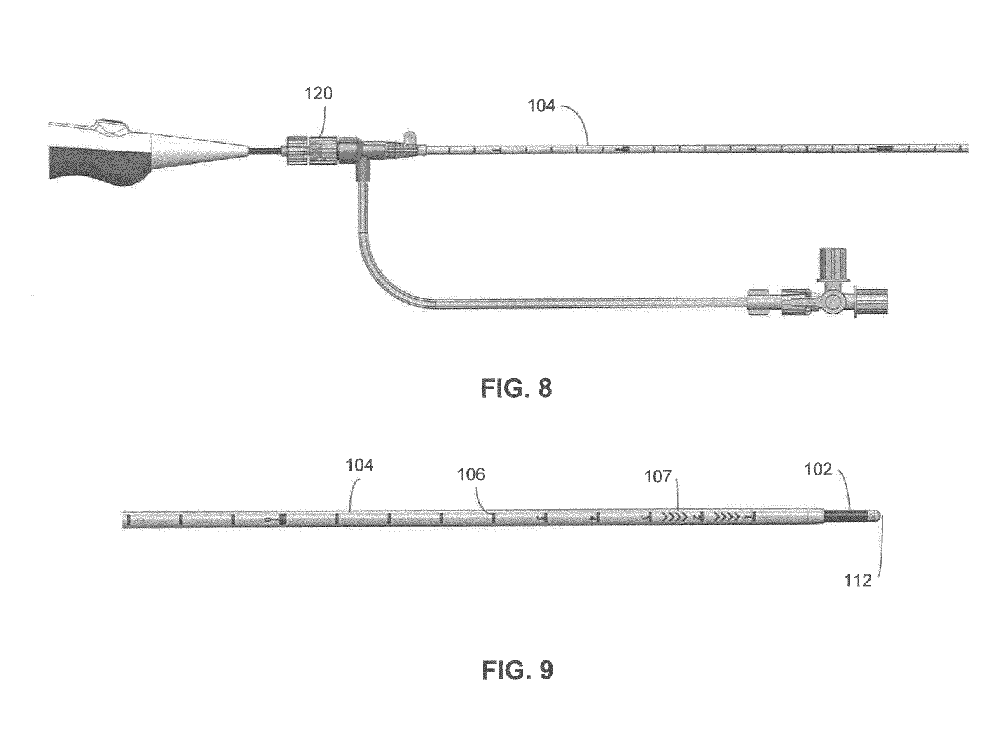

FIGS. 6-9 show an embodiment of the invention in which the catheter shaft 102 has a fitting 120 that mates with the sheath 104 to maintain the relative positions of the shaft 102 and sheath 104. As shown in the detail of FIG. 9, when the proximal end of the sheath 104 engages fitting 120, the distal tip 112 of the catheter shaft 102 is exposed for vapor delivery. After the sheath 104 mates with the fitting 120 on shaft 102, catheter shaft 102 and sheath 104 can be retracted proximally as a unit. In some embodiments, the location of the fitting 120 on catheter shaft 102 can be adjusted to change the amount the distal end 112 of shaft 102 extends from the distal end of the sheath 104 when sheath 104 mates with fitting 120.

FIG. 9 also shows an optional feature in which the sheath markings 106 include markings 107 at the distal end of sheath 104 to indicate to the user when the sheath is about to emerge from the patient's vein access point. This feature helps avoid unintentional removal of the vapor delivery catheter from the patient while vapor is still being delivered. This feature also lets the user know how much more of the catheter is still in the patient, i.e., how much further the user will need to remove the catheter before it is completely removed from the vein access point.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of delivering therapy to a vein, comprising:
inserting a structural sheath into the vein, the structural sheath being configured to prevent collapse of the vein due to spasm or via the administration of tumescent anesthesia;
advancing a vapor delivery shaft into the catheter sheath;
positioning a vapor delivery tip of the vapor delivery shaft distally of the catheter sheath; and
delivering vapor to the vein through the vapor delivery tip.

2. The method of claim 1 wherein the vapor is generated remotely from the vapor delivery shaft.

3. The method of claim 1 wherein the vapor is generated within the vapor delivery shaft.

4. The method of claim 1 further comprising, prior to the delivering step, retracting the structural sheath proximally along the vapor delivery shaft to expose a portion of the vapor delivery shaft.

5. The method of claim 4 wherein the portion of the vapor delivery shaft comprises a hot zone having a length of approximately 5 cm to 15 cm.

6. The method of claim 4 wherein the retracting step comprises retracting the structural sheath until a portion of the sheath engages a fitting extending from the vapor delivery shaft.

7. The method of claim 1 further comprising pulling the structural sheath and the vapor delivery shaft proximally along the vein during the delivering vapor step.

8. The method of claim 7 further comprising maintaining a relative position between the catheter sheath and the vapor delivery shaft during the pulling step.

9. The method of claim 1 further comprising shrinking the vein with the vapor.

10. A method of treating a vessel, comprising:
inserting a structural sheath into a vessel to be treated;
applying tumescent anesthesia around the vessel;
preventing the vessel from collapsing due to the tumescent anesthesia with the structural sheath;
advancing a vapor delivery catheter into the sheath to position delivery vapor ports of the catheter within the vessel; and
delivering vapor through the vapor delivery ports to treat the vessel.

11. The method of claim 10 wherein the vapor shrinks the vessel.

12. The method of claim 10 further comprising pulling the structural sheath and the vapor delivery catheter proximally within the vein during the delivering step.

13. The method of claim 12 further comprising:
stopping the pulling of the structural sheath and the vapor delivery catheter when the catheter reaches a feeder vein to be treated;
steering the delivery vapor ports into or towards the feeder vein; and
delivering vapor through the vapor delivery ports into the feeder vein to treat the feeder vein.

14. A vapor delivery catheter system comprising:
a structural sheath adapted to be inserted into a vein; and
a vapor delivery shaft adapted to be surrounded by the structural sheath, the vapor delivery shaft comprising a vapor delivery port at a distal end of the shaft and a fitting at a proximal end of the shaft, the fitting being adapted to engage with the structural sheath when the vapor delivery port extends from a distal end of the sheath.

15. The system of claim 14 wherein the fitting is further adapted to attach the sheath to the vapor delivery shaft so that the sheath and the shaft can be moved as a unit.

16. The system of claim 14 wherein the sheath has markings at the distal end of the sheath adapted to alert the user when the sheath is about to exit the entry site.

17. The sheath of claim 16 wherein the markings comprise regularly spaced markings adapted to indicate depth of insertion of the sheath and at least one distal marking distinct from the regularly spaced markings at the distal end of the sheath.
